(19) United States
(12) Patent Application Publication
(10) Pub. No.: US 2024/0331810 A1
(43) Pub. Date: Oct. 3, 2024

(54) COMPOSITION EVALUATION METHOD, COMPOSITION EVALUATION DEVICE, COMPOSITION, AND COMPOSITION MANUFACTURING METHOD

(71) Applicant: Meiji Co., Ltd., Tokyo (JP)

(72) Inventors: Hayate Yamanaka, Tokyo (JP); Akihiro NAKAJIMA, Tokyo (JP); Masayo TAKAHASHI, Tokyo (JP); Kai NAKAYAMA, Tokyo (JP); Shintarou NANAUMI, Tokyo (JP)

(21) Appl. No.: 18/616,645

(22) Filed: Mar. 26, 2024

(30) Foreign Application Priority Data
Mar. 31, 2023 (JP) 2023-059314
Feb. 27, 2024 (JP) 2024-027851

(51) Int. Cl. G16C 20/30; G16C 20/70

(52) U.S. Cl. CPC G16C 20/30; G16C 20/70

(57) ABSTRACT

A composition evaluation device included an analyzer that analyzes a plurality of contained components that are contained or estimated to be contained in a composition and also analyzes component amounts of the plurality of contained components, a molecular descriptor calculator that calculates molecular descriptors related to chemical structures of the contained components, an activity value estimator that estimates activity values of the contained components, based on the molecular descriptors, and a score calculator that calculates a total activity score of the plurality of contained components from the estimated activity values of the contained components and the component amounts.

### CROSS-REFERENCE TO RELATED APPLICATIONS

This application is based upon and claims the benefit of priority of the prior Japanese Patent Application No. 2023-059314, filed on Mar. 31, 2023, and the Japanese Patent Application No. 2024-027851 filed on Feb. 27, 2024 the entire contents of which are incorporated herein by reference.

### FIELD

The present invention relates to a composition evaluation method, a composition evaluation device, a composition, and a composition manufacturing method.

### BACKGROUND

In recent years, the functions of compositions, for example, the functions of foods have attracted attention. The functions include, for example, functions related to nutrition, functions related to preference, and functions related to biological regulation. In particular, foods that have functions related to nutrition and biological regulation are attracting attention from the viewpoint of maintaining health or treating diseases and injuries.

Foods that have certain functions are in some cases called functional foods, for example. Among functional foods, foods with functional claims are foods of which the functionality based on scientific evidence is indicated on the responsibility of the business operator. r. With regard to foods with functional claims, the specific health purposes, for example, “suppressing the rise in blood sugar levels after meals” and “regulating stomach health” expected by consuming the food are indicated on the food package. For example, a consumer refers to this indication and purchases a food that has a specific health purpose (effect, function) desired by the consumer.

For example, foods and drinks containing Lactococcus lactis strain Plasma are labeled “it has been reported that the foods act on pDC (plasmacytoid dendritic cells) and help to maintain immune function in healthy people”, and are produced and sold as foods with functional claims.

With regard to the foods with functional claims, technologies related to an IFN inducer that contains lactic acid bacteria as an active component and is capable of inducing IFN production, an immunoenhancing agent or a virus infection protective agent containing the inducer, and a food or drink that contains the inducer and has IFN inducing activity, immunoenhancing activity, or virus infection protective activity are disclosed in Japanese Patent Application Publication No. 2017-201984 and WO 2012/091081.

### SUMMARY

As described above, the indicated functions of a composition have been examined based on the content and intake of a specific responsible component in the composition.

Foods containing a large amount of y-aminobutyric acid (hereinafter referred to as GABA) are labeled, for example, “it has been reported that the foods have a function of helping to improve sleep quality (falling asleep, depth of sleep, waking up refreshed)”. Foods containing a large amount of gallate-type catechins are labeled, for example, “it has been reported that a function of reducing body fat and LDL cholesterol is elicited”.

However, foods containing a large amount of GABA (excluding supplements of which the main components are components involved in the function) in some cases contain components that inhibit the function of “helping to improve sleep quality (falling asleep, depth of sleep, waking up refreshed)”.

In this case, a consumer will not experience as much effect as expected when consumes this food.

For example, a certain food does not contain GABA but may contain multiple types of components that have the function of “helping to improve sleep quality (falling asleep, depth of sleep, waking up refreshed)”. However, in a case where each of the multiple types of components is less effective in “helping to improve sleep quality (falling asleep, depth of sleep, waking up refreshed)” than GABA, the certain food is in some cases not evaluated as a food that “helps to improve sleep quality (falling asleep, depth of sleep, waking up refreshed)” although the certain food has the function of “helping to improve sleep quality (falling asleep, depth of sleep, waking up refreshed)”.

As described above, when the function of a food is evaluated based on whether the food contains one responsible component, it is not possible to accurately evaluate the function of the food in some cases. Hence, it is necessary to consider the influences of multiple components to accurately evaluate the functionality of foods.

However, not all of the functions of components contained in foods have been elucidated to date. A huge amount of analysis and calculation is required to accurately evaluate the functions of a food by taking all the components of the food into consideration, and it is difficult to realize the accurate evaluation in terms of time and cost.

Accordingly, this disclosure provides a composition evaluation method, a composition evaluation device, a composition, and a composition manufacturing method by which proper evaluation of functions of foods can be efficiently implemented.

The present invention relates to a composition evaluation device comprising: an analyzer that analyzes a plurality of contained components that are contained or estimated to be contained in a composition and also analyzes component amounts of the plurality of contained components; a molecular descriptor calculator that calculates molecular descriptors related to chemical structures of the contained components; an activity value estimator that estimates activity values of the contained components, based on the molecular descriptors; and a score calculator that calculates a total activity score of the plurality of contained components from the estimated activity values of the contained components and the component amounts, wherein the activity value estimator estimates activity values of the contained components by a learned model that has learned using a feature quantity selected from the molecular descriptors for known components and a corresponding activity value as training data.

The present invention relates to a composition evaluation device in which the learned model detects a component having a chemical structure, which is similar to a chemical structure of the contained component, from learned components, and calculates an activity value of the detected component as an activity value of the contained component.

The present invention relates to a composition evaluation device in which the total activity score is a logarithm of a sum of values obtained by dividing concentrations of respective contained components by activity values.

The present invention relates to a composition evaluation device in which the total activity score is a value obtained by summing numerical values obtained by multiplying activity values of the plurality of contained components by component amounts of the plurality of contained components.

The present invention relates to a composition evaluation device in which the learned model has learned using the feature quantity of a chemical structure of a component and the activity value, and the feature quantity includes a feature quantity related to a sum of surface areas of atoms. The present invention relates to a composition evaluation device in

which the learned model has learned using the feature quantity of a chemical structure of a component and the activity value, and the feature quantity includes a feature quantity related to a sum of electron distributions of atoms.

The present invention relates to a composition evaluation device in which the learned model has learned using the feature quantity of a chemical structure of a component and the activity value, and the feature quantity includes a feature quantity related to an atom-based Log P index.

The present invention relates to a composition evaluation device in which the learned model has learned using the feature quantity of a chemical structure of a component and the activity value, and the feature quantity includes a feature quantity related to eigenvalues in a case where atoms constituting one compound and bonds between atoms are expressed in a matrix and diagonal components are taken as atomic weights.

The present invention relates to a composition evaluation device in which the learned model has learned using the feature quantity of a chemical structure of a component and the activity value, and the feature quantity includes a feature quantity related to the number of hydrogen bond donors.

The present invention relates to a composition evaluation device in which the known component includes the contained component estimated using the learned model and the corresponding activity value includes an activity value of the contained component estimated using the learned model.

The present invention relates to a composition evaluation device in which the contained component is a peptide, the activity value is a half maximal inhibitory concentration for ACE (angiotensin-I converting enzyme), the known component is a peptide of which an activity value is known, and the score calculation unit calculates the total activity score, based on the calculated half maximal inhibitory concentration for ACE.

The present invention relates to a composition evaluation method including an analysis step of analyzing a plurality of contained components that are contained or estimated to be contained in a composition and also analyzing component amounts of the plurality of contained components, a descriptor calculation step of calculating molecular descriptors related to chemical structures of the contained components, an estimation step of estimating activity values of the contained components, based on the molecular descriptors, and a score calculation step of calculating a total activity score of the plurality of contained components from the estimated activity values of the contained components and the component amounts, in which activity values of the contained components are estimated using a learned model that has learned using a feature quantity selected from the molecular descriptors for known components and a corresponding activity value as training data in the estimation step.

The present invention relates to a composition manufactured based on evaluation by a total activity score, in which the total activity score is calculated from estimated activity values of a plurality of contained components and component amounts, which are obtained by analyzing the plurality of contained components that are contained or estimated to be contained in a composition and the component amounts of the plurality of contained components, calculating molecular descriptors related to chemical structures of the contained components, and estimating activity values of the contained components, based on the molecular descriptors.

The present invention relates to a composition manufacturing method for manufacturing a composition, based on evaluation by a total activity score, in which the total activity score is calculated from estimated activity values of a plurality of contained components and component amounts, which are obtained by analyzing the plurality of contained components that are contained or estimated to be contained in a composition and the component amounts of the plurality of contained components, calculating molecular descriptors related to chemical structures of the contained components, and estimating activity values of the contained components, based on the molecular descriptors.

By a disclosure, it is possible to efficiently implement proper evaluation of functions of compositions.

### BRIEF DESCRIPTION OF DRAWINGS

FIG. 1 is a diagram illustrating a configuration example of a food evaluation system 10.

FIG. 2 is a diagram illustrating a configuration example of the food evaluation device 100.

FIG. 3 is a diagram illustrating an example of the concept of a total activity score.

FIG. 4 is a diagram illustrating an example of software configuration in food evaluation processing.

FIG. 5 is a diagram illustrating an example of software configuration in model training processing.

FIG. 6 is a diagram illustrating an example of a processing flowchart of food evaluation processing S100.

FIG. 7 is a diagram illustrating an example of a processing flowchart of the component analysis processing S102.

FIG. 8 is a diagram illustrating an example of a processing flowchart of the molecular descriptor calculation processing S104.

FIG. 9 is a diagram illustrating an example of feature quantities to be calculated.

FIG. 10 is a diagram illustrating an example of a processing flowchart of the estimated activity value calculation processing S105.

FIG. 11 is a diagram illustrating an example of a processing flowchart of the total activity score calculation processing S106.

FIG. 12 is a diagram illustrating an example of equations for calculating the total activity score.

FIG. 13 is a diagram illustrating an example of a processing flowchart of model training processing S200.

FIG. 14 is a diagram illustrating an example of the total activity scores of a food A and a food B.

### DESCRIPTION OF EMBODIMENTS

### First Embodiment

A first embodiment will be described.

The food evaluation described below is an example of composition evaluation, and “food” in the text can be replaced with “composition”.

Composition evaluation also includes evaluation of herbal medicines, and “composition” and “food” in the text can be replaced with “herbal medicines”. Furthermore, the composition is not limited to foods and herbal medicines. Configuration Example of Food Evaluation System 10

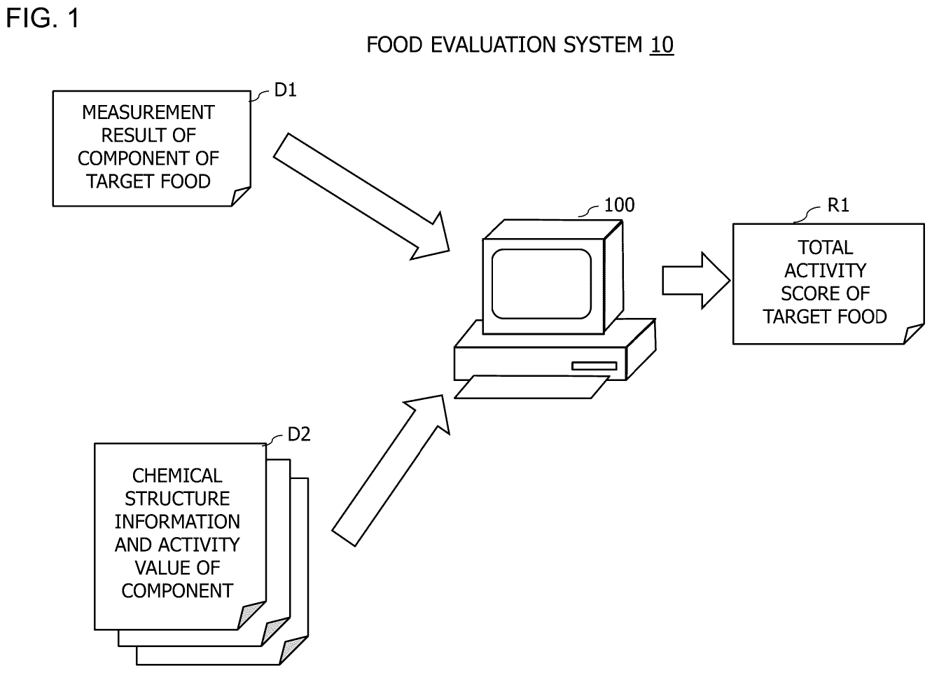

FIG. 1 is a diagram illustrating a configuration example of a food evaluation system 10. The food evaluation system 10 includes a food evaluation device 100. The food evaluation system 10 receives data D1 of the measurement results of the components of a target food to be evaluated input into the food evaluation device 100 and outputs a total activity score R1 of the target food. The food evaluation system 10 receives and acquires a data group D2 consisting of chemical structure information of known components and activity values corresponding thereto. The food evaluation device 100 stores the data group D2, and calculates feature quantities of the known components from the chemical structure information of the known components. The food evaluation device 100 uses the feature quantities of the known components and the activity values corresponding thereto as training data to train a learning model (estimated activity value calculation model) that is AI (artificial intelligence) and construct a learned model.

The chemical structure information is information about chemical structures, and includes, for example, information about notations that makes it possible to specify molecular structures, such as one-letter notation, three-letter notation, and SMILES notation for amino acids. The chemical structure information may include, for example, information that makes it possible to specify chemical structures, such as component names and chemical formulas.

The data group D2 is a data group indicating a plurality of data corresponding to a plurality of known components. The data group D2 indicates a data group extracted from existing literatures, public databases, experimental results, and the like.

The food evaluation device 100 acquires the data D1 and calculates and outputs the total activity score R1 of the target food. The food evaluation device 100 outputs data by, for example, converting the data into a file or displaying the data on a screen.

The food evaluation device 100 searches for the chemical structure of a component included in the data D1 from the chemical structures of the stored components. Then, the food evaluation device 100 compares the chemical structure of the component included in the data D1 with the chemical structures of components, which are included in the data group D2 and have known activity values, to estimate the activity value of the component included in the data D1 from the degree of similarity of chemical structure. Then, the food evaluation device 100 calculates the total activity score of the target food from the activity value estimated for each component included in the data D1.

The total activity score is a numerical value by taking the activity values of a plurality of components contained in the target food into consideration, and is, for example, the sum of the numerical values obtained by multiplying the component amounts by the activity values for individual components. Estimation of the activity value of each component is performed by a learned estimated activity value calculation model.

The activity value indicates, for example, the degree or amount of effect or influence on living organisms (for example, humans). The effect and influence include both good ones and bad ones. Examples of the activity value include IC₅₀ (half maximal (50%) inhibitory concentration), EC₅₀ (half maximal (₅₀%) effective concentration), pD₂ (index of efficacy), pA₂ (activity indicator of competitive antagonist), pD′₂ (activity indicator of non-competitive antagonist), ED₅₀ (median effective dose), LD₅₀ (median lethal dose), NOEL (no observed effect level), NOAEL (no observed adverse effect level), LOAEL (lowest observed adverse effect level), BMDL (benchmark dose lower confidence limit), ADI (acceptable daily intake), and ARfD (acute reference dose). Substances (components) that contribute to the activity value may be referred to as biologically active substances, for example. The biologically active substances include, for example, not only substances that regulate specific physiological functions of the human body but also substances that inhibit specific functions. The composition to be evaluated is not particularly limited, and

includes, for example, foods and herbal medicines. The foods include, for example, fresh foods and processed foods that contain a plurality of components, and also include food additives and seasonings used by being added to foods. The herbal medicines include, for example, processed ones derived from animals and plants that contain a plurality of components, and also include extracts of these.

### Configuration Example of Food Evaluation Device 100

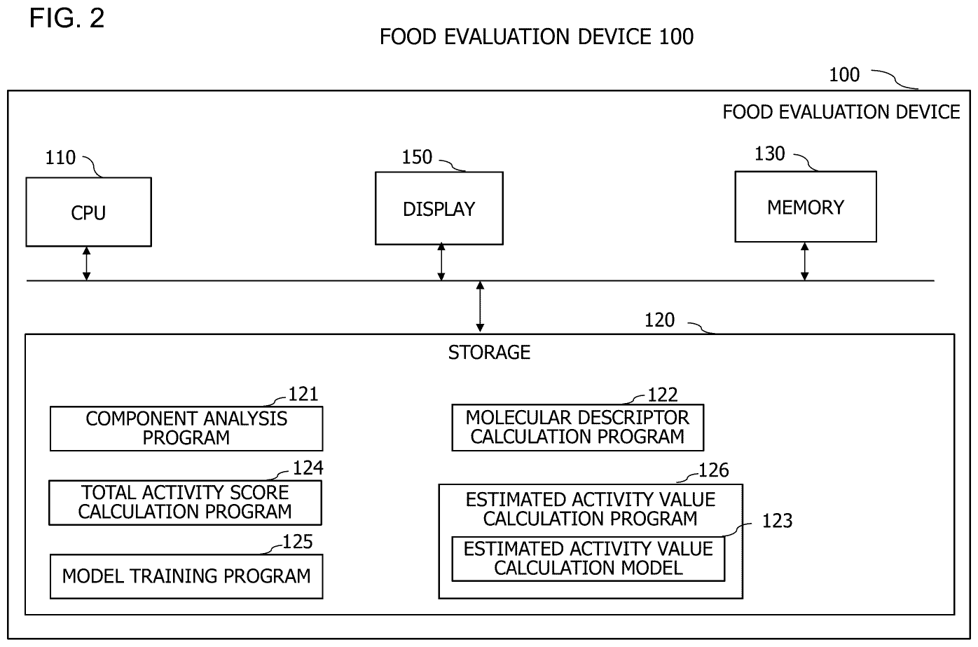

FIG. 2 is a diagram illustrating a configuration example of the food evaluation device 100. The food evaluation device 100 includes a CPU (central processing unit) 110, a storage 120, a memory 130, and a display 150.

The storage 120 is an auxiliary storage, such as a flash memory, a HDD (hard disk drive), or a SSD (solid state drive), which stores programs and data. The storage 120 stores a component analysis program 121, a molecular descriptor calculation program 122, an estimated activity value calculation program 126, a total activity score calculation program 124, and a model training program 125.

The memory 130 is a region into which programs stored in the storage 120 are loaded. The memory 130 may also be used as a region for programs to store data.

The display 150 is a screen that displays the total activity score, input/output data, and the like, and is a display section. The display 150 may be, for example, an integrated one or externally connected one.

The CPU 110 is a processor that loads programs stored in the storage 120 into the memory 130, executes loaded programs, constructs each section, and implements each step of processing.

The CPU 110 executes the component analysis program 121 to construct a component analysis section and perform component analysis processing. The component analysis processing is processing of calculating the components contained in the target food, the amounts (proportions) of the components, and the like from the component measurement data of the target food. In the component analysis processing, for example, the components of the target food are specified and the chemical structures of the specified components are specified.

The CPU 110 executes the molecular descriptor calculation program 122 to construct a molecular descriptor calculation section and perform molecular descriptor calculation processing. In the molecular descriptor calculation processing, molecular descriptors are calculated from the chemical structures of the components contained in the target food. The molecular descriptor is, for example, a feature of a chemical structure calculated from chemical structure information, and is physicochemical information calculated from the molecular structure. For example, about 2000 types of molecular descriptors are calculated in the molecular descriptor calculation processing.

The CPU 110 executes the total activity score calculation program 124 to construct a total activity score calculation section (score calculation section) and performs the total activity score calculation processing. The total activity score calculation processing is processing of calculating the total activity score of the target food from the estimated activity values of the respective components.

The CPU 110 executes the model training program 125 to construct a model training section and perform model training processing. The model training processing is processing of inputting training data into an estimated activity value calculation model 123 and training the model.

The CPU 110 executes the estimated activity value calculation program 126 to construct an estimated activity value calculation section and perform estimated activity value calculation processing. The estimated activity value calculation processing is processing of estimating the activity value of a component based on the chemical structure information of the component. In the estimated activity value calculation processing, the estimated activity value calculation model 123 is used to calculate the estimated activity value of a component.

The estimated activity value calculation model 123 is, for example, AI, and estimates the activity value of an input component. The estimated activity value calculation model 123 is constructed by, for example, executing a specific program on the CPU 110. The estimated activity value calculation model 123 compares the chemical structure of an input component with the chemical structure of a learned component to determine similarity. Then, the estimated activity value calculation model 123 estimates the activity value of the input component according to the similarity.

The estimated activity value calculation model 123 may detect (search for) a learned component having a chemical structure similar to the chemical structure of the input component, and calculate the activity value of the detected component as the activity value of the input component.

The estimated activity value calculation model 123 learns chemical structure information and activity values of components of which the activity values are known to be a learned model. The estimated activity value calculation model 123 performs learning using, for example, feature quantities (or molecular descriptors) related to chemical structures and activity values as training data.

### About Total Activity Score

The total activity score will be described. The total activity score is not an evaluation value of a food by one responsible component, but a comprehensive evaluation value by a plurality of components contained in the food.

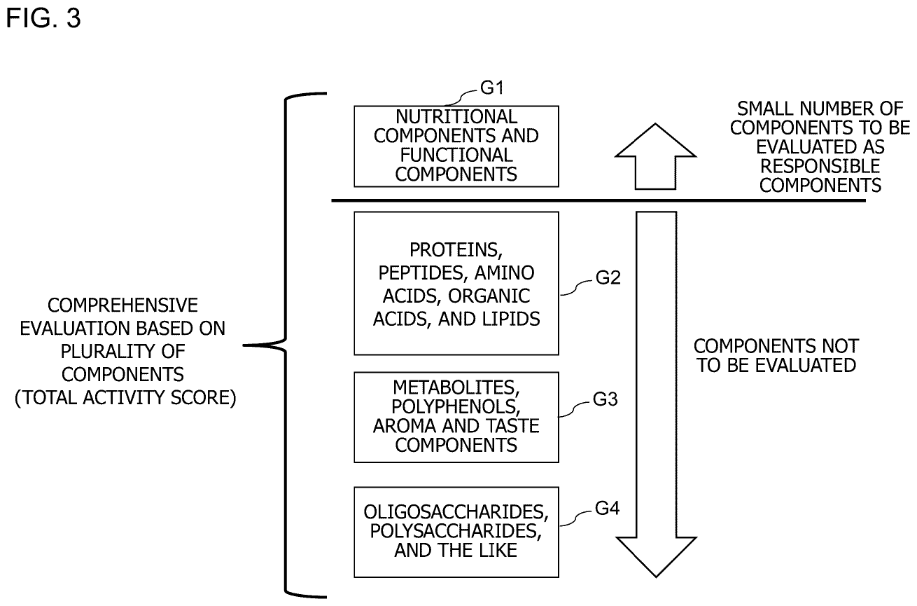

FIG. 3 is a diagram illustrating an example of the concept of a total activity score. The nutritional components and functional components included in a component group G1 are known components and are used in food evaluation as one of the responsible components. An example of a component included in the component group G1 is GABA, which is known as a component having an act of helping to improve sleep quality (falling asleep, depth of sleep, waking up refreshed). As for food evaluation by one responsible component, a food is evaluated as a food that exerts an act of helping to improve sleep quality (falling asleep, depth of sleep, waking up refreshed) when the GABA content in the food is a certain amount or more.

However, in this evaluation method, the activity values (effects on the body) of components other than the responsible component are not evaluated, and it is thus not possible to properly evaluate foods in some cases. Therefore, the food evaluation system 10 performs food evaluation by the total activity score obtained by taking a plurality of components (preferably all components) into consideration.

The total activity score is a value obtained by taking not only the component group G1 but also component groups G2 to G4 into consideration. For example, an example of the total activity score for “preventive effect on a disease X” will be described. For example, it is assumed that a certain food Q contains at least a component a belonging to the component group G1, a component b belonging to the component group G2, a component c belonging to the component group G3, and a component d belonging to the component group G4.

For example, the component a belonging to the component group G1 is a component that is not approved to be effective in prevention of the disease X. In this case, if the evaluation is evaluation by one responsible component, the food will be evaluated not to have a preventive effect on the disease X.

However, there is a case where each of the component b, component c, and component d has the effect of suppressing the onset of the disease X, albeit to a small extent. Although the effect of each component individually is small, the effect becomes greater as the total amount of the three components increases. Hence, in the evaluation using the total activity score, the food Q is evaluated to have a preventive effect on the disease X depending on the total amount of the component b, component c, and component d.

The cause of the disease X is often not a single factor but a combination of a plurality of factors. For example, dementia is a general term for a plurality of diseases such as Alzheimer's disease and cerebrovascular dementia. Causes of cerebrovascular dementia include, for example, arteriosclerosis and decreased cerebral blood flow. Furthermore, causes of arteriosclerosis include, for example, hypertension and diabetes.

In order to prevent dementia, for example, it is effective to prevent arteriosclerosis. Meanwhile, prevention of hypertension, which is a cause of arteriosclerosis, also has an indirect, but preventive effect on dementia.

Therefore, the food evaluation system 10 may also consider indirect effects (activity values) in the calculation of total activity score. For example, there is a case where a component a has a direct preventive effect on a disease X and a component b, a component c, and a component d have indirect preventive effects on the disease X. In this case, the total activity score is a value obtained by adding the activity values of the component b, component c, and component d to the activity value of the component a. The total activity score may use coefficients to change the weights of the component a having a direct effect and the component b, component c, and component d having indirect effects. For example, when the total activity score is the sum of the activity values of the respective components, the component b, component c, and component d are multiplied by a coefficient lower than that for the component a and added.

For example, there is a case where a component d has an adverse effect on the disease X, such as inhibition of the activity of a component that prevents arteriosclerosis. In this case, in the calculation of total activity score, the component d is calculated as a negative value.

As described above, the total activity score is a value obtained by comprehensively evaluating the activity values of a plurality of components contained in a food, and is a value indicating the degree of effect on the human body. For example, as the total activity score is larger, the effect is greater.

The total activity score can more properly evaluate the effects of a food by comprehensively evaluating a plurality of components in a case where the respective components contained in a food singly have weak functionality (effect) as well.

The total activity score can indicate, for example, the degree of preventive effect on each of a plurality of diseases. For example, medical professionals and food companies can appropriately respond to the requests of users and patients by proposing food combinations obtained by taking the total activity score into consideration or planning and manufacturing foods obtained by taking the total activity score into consideration for users and patients who want to prevent a plurality of diseases.

The components contained in a food to be analyzed are not particularly limited, but include, for example, components belonging to categories such as proteins, peptides, amino acids, organic acids, lipids, polysaccharides, oligosaccharides, saccharides, vitamins, minerals, polyphenols, alkaloids, carotenoids, terpenes, metabolites, aroma components, and taste components.

### Software Configuration Example of Food Evaluation Device 100

The food evaluation device 100 includes a component analysis unit 20, a molecular descriptor calculation unit 21, an estimated activity value calculation unit 22, a total activity score calculation unit 23, and a model training unit 24. Hereinafter, an overview of the processing of each unit and model in the food evaluation processing and model training processing will be described.

### 1. Software Configuration in Food Evaluation Processing

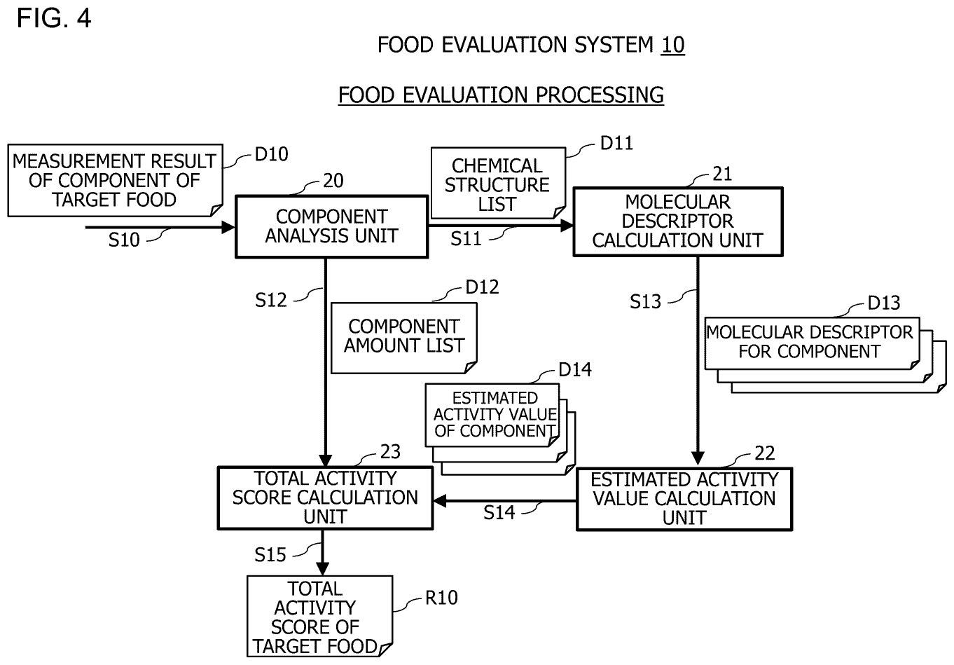

FIG. 4 is a diagram illustrating an example of software configuration in food evaluation processing. The food evaluation processing is processing of outputting the total activity score of a target food.

The component analysis unit 20 acquires a component measurement result D10 of a target food (S10). The component measurement result D10 includes, for example, raw data (for example, LC-MS/MS data) from which chemical structures and component amounts can be calculated and processed data (for example, amino acid sequence information) including data related to component names and chemical structures. The component analysis unit 20 analyzes the names of components contained in the target food and the chemical structures of the components from the component measurement result D10 and acquires or calculates (estimates) the amount of each component (hereinafter referred to as component amount in some cases). Then, the component analysis unit 20 outputs a chemical structure list (a list presenting component names and chemical structures of the components) D11 to the molecular descriptor calculation unit 21 (S11). The component analysis unit 20 also outputs a component amount list D12 including component names and component amounts to the total activity score calculation unit 23 (S12).

The molecular descriptor calculation unit 21 acquires the chemical structure list D11 (S11), and calculates a molecular descriptor from the chemical structure of each component included in the chemical structure list D11. For example, the molecular descriptor calculation unit 21 searches for the component from stored information in which a component and a molecular descriptor for the component are stored in association with each other, and calculates the component as a molecular descriptor. The molecular descriptor calculation unit 21 also calculates a molecular descriptor related to the chemical structure for every component. Then, the molecular descriptor calculation unit 21 outputs a data group D13 of molecular descriptors for the components to the estimated activity value calculation unit 22 (S13).

Upon acquiring the data group D13 (S13), the estimated activity value calculation unit 22 calculates the estimated activity value of each component. The estimated activity value calculation unit 22 assumes, for example, that components having similar chemical structures exhibit similar biological activities to estimate the activity value of each component. The estimated activity value calculation unit 22 outputs a data group D14 of the estimated activity values of components to the total activity score calculation unit 23 (S14). The estimated activity value calculation unit 22 uses a learned model (estimated activity value calculation model) to calculate the estimated activity value.

The estimated activity value calculation unit 22 may, for example, extract molecular descriptors of the same type as the feature quantities used for training and use the molecular descriptors to estimate the activity values. The estimated activity value calculation unit 22 may use molecular descriptors of the same type as the feature quantities used for training and molecular descriptors other than these (for example, all molecular descriptors) to estimate the activity values.

Upon acquiring the data group D14 (S14), the total activity score calculation unit 23 calculates the total activity score of the target food based on the activity values of the respective components in the data group D14 and the component amount list D12 of the components acquired from the component analysis unit 20. Then, the total activity score calculation unit 23 outputs a calculated total activity score R10 to a screen, a file, and the like (S15).

### 2. Software Configuration in Model Training Processing

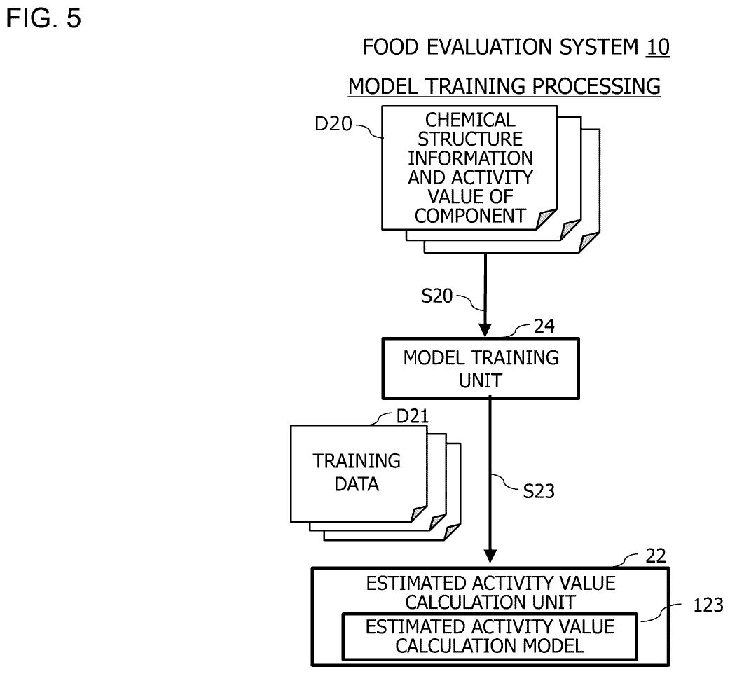

FIG. 5 is a diagram illustrating an example of software configuration in model training processing. The model training processing is processing of training the estimated activity value calculation model 123 of the estimated activity value calculation unit 22.

The model training unit 24 acquires a data group D20 of chemical structure information of a component (a component of which the activity value is known) and the activity value of the component (S20). The model training unit 24 extracts the chemical structure of the component from the data group D20. The model training unit 24 then calculates a molecular descriptor from the chemical structure of the component.

The model training unit 24 selects a molecular descriptor to be used for training from the calculated molecular descriptors as a feature quantity. Then, the model training unit 24 uses the feature quantity of a component and the corresponding activity value as training data to output a training data group D21 to the estimated activity value calculation unit 22 having the estimated activity value calculation model 123 (S23).

The estimated activity value calculation model 123 of the estimated activity value calculation unit 22 receives the training data group D21 (S23), and learns the feature quantity of the chemical structure and the activity value for every component to be a learned model.

### Details of Each Step of Processing

Details and examples of each step of processing will be described.

### 1. Food Evaluation Processing

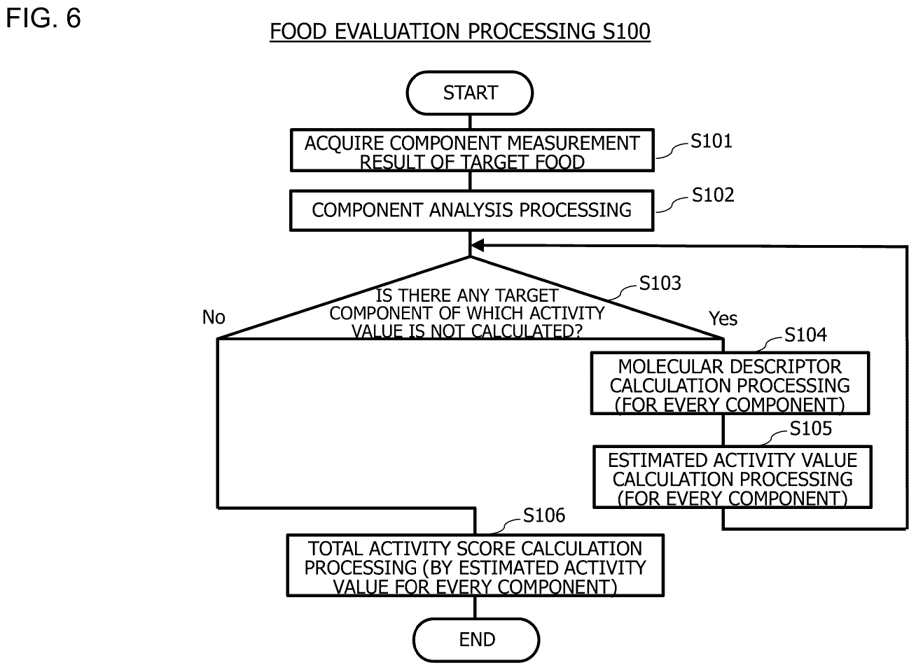

FIG. 6 is a diagram illustrating an example of a processing flowchart of food evaluation processing S100. The food evaluation device 100 acquires the component measurement results of a target food (S101).

The food evaluation device 100 performs component analysis processing (S102).

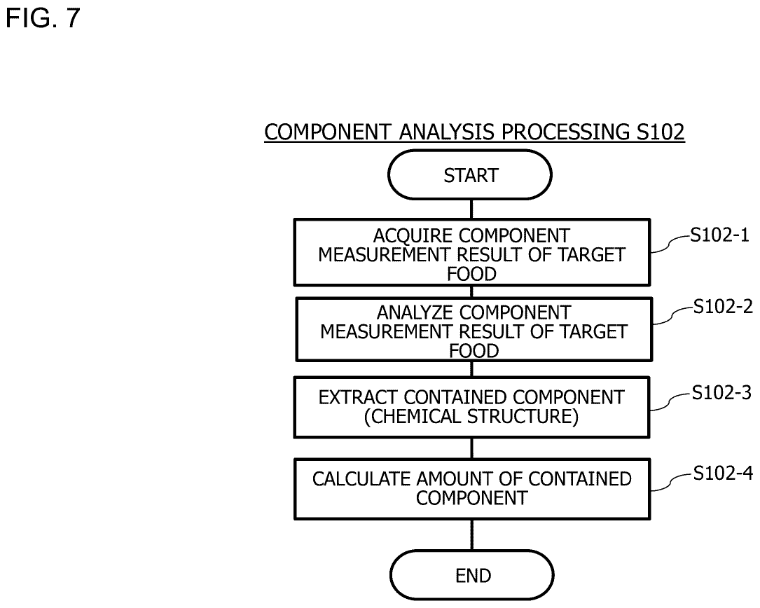

FIG. 7 is a diagram illustrating an example of a processing flowchart of the component analysis processing S102. The food evaluation device 100 acquires the component measurement results of the target food (S102-1).

The food evaluation device 100 analyzes the component measurement results of the target food (S102-2). The analysis is processing of calculating (estimating) the types (chemical structures) of components and the component amounts.

As a result of the analysis, the food evaluation device 100 extracts the contained components (chemical structures) (S102-3), further calculates the component amounts of the contained components (S102-4), and ends the processing.

For example, an example of calculating the total activity score of the half maximal inhibitory concentration IC₅₀ (the concentration at which the action of ACE (angiotensin-I converting enzyme) is inhibited by 50%) for ACE (hereinafter, this example is referred to as an example of ACE inhibitory activity in some cases) will be described. In the example of ACE inhibitory activity, the target component is a peptide and components other than this are not considered. Component analysis in the example of ACE inhibitory activity includes peptidome analysis. By performing peptidome analysis, the types of peptides estimated to be contained and the estimated component amount of each peptide are calculated. The component amount is calculated using, for example, the peak area of XIC (extracted ion chromatogram) or the total peak area of TIC (total ion chromatogram) by LC-MS/MS.

Returning to the processing flowchart of FIG. 6, the food evaluation device 100 performs molecular descriptor calculation processing on all extracted components (S103: Yes) (S104) and further performs estimated activity value calculation processing (S105).

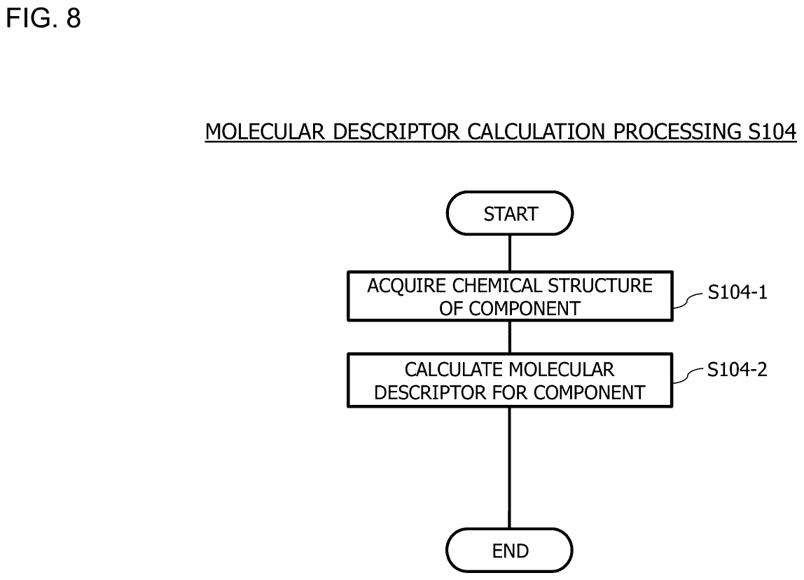

FIG. 8 is a diagram illustrating an example of a processing flowchart of the molecular descriptor calculation processing S104. The food evaluation device 100 acquires the chemical structure of a component for which a molecular descriptor is to be calculated (S104-1).

The food evaluation device 100 calculates the molecular descriptor for the component from the acquired chemical structure (S104-2), and ends the processing.

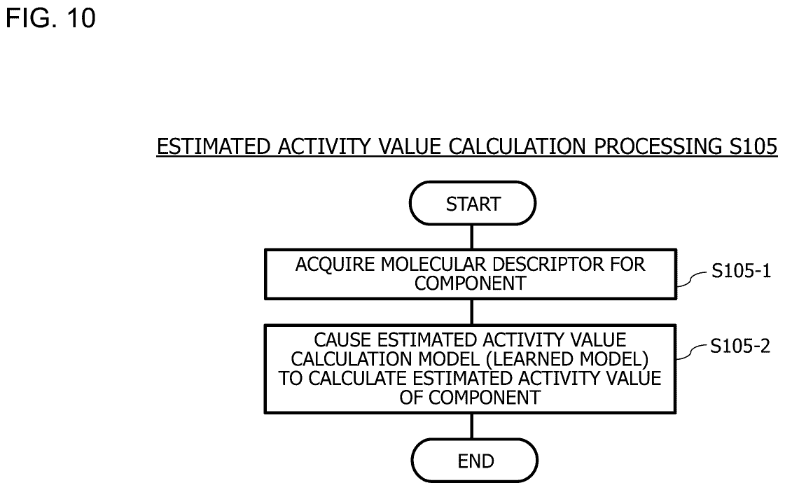

FIG. 10 is a diagram illustrating an example of a processing flowchart of the estimated activity value calculation processing S105. The food evaluation device 100 acquires the molecular descriptor for a component (S105-1).

The food evaluation device 100 inputs the molecular descriptor for the component into the estimated activity value calculation model (learned model), causes the estimated activity value calculation model to calculate the estimated activity value of the component (S105-2), and ends the processing. The estimated activity value calculation model has learned and can calculate the estimated activity value from the molecular descriptor for the component.

In the example of ACE inhibitory activity, the estimated activity value calculation model calculates the estimated activity value according to the type of peptide. This estimated activity value is an activity value estimated by a model that has learned using peptides and other components having ACE inhibitory activity as training data. The feature quantities used in the training data will be described below.

Returning to the processing flowchart of FIG. 6, when the calculation of estimated activity values for all components is completed (No in S103), total activity score calculation processing is performed (S106), and the processing ends.

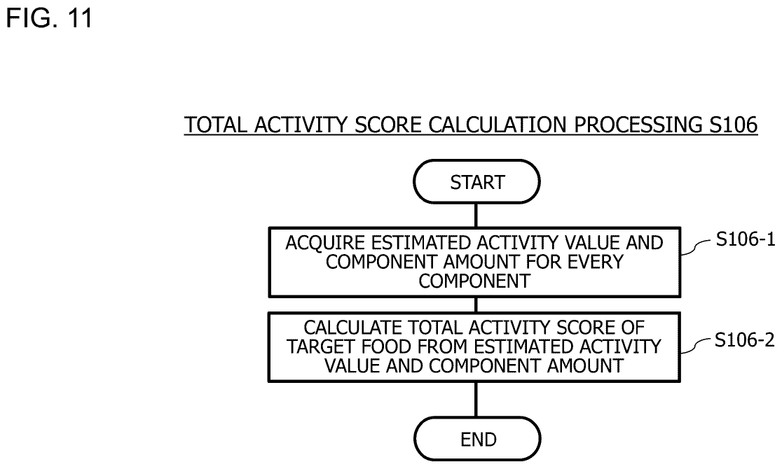

FIG. 11 is a diagram illustrating an example of a processing flowchart of the total activity score calculation processing S106. The food evaluation device 100 acquires the estimated activity value and component amount for every component (S106-1). The food evaluation device 100 calculates the total activity score of the target food from the acquired estimated activity value and component amount for every component (S106-2), and ends the processing.

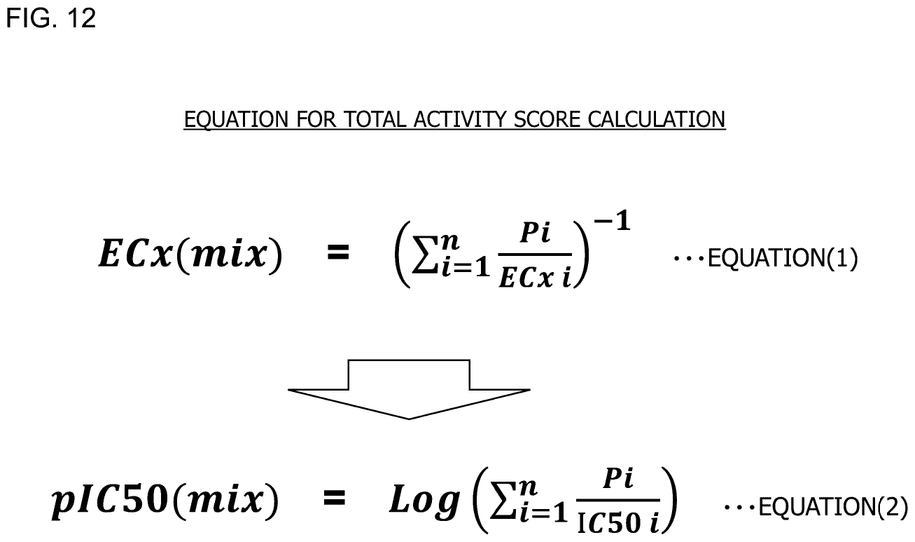

An example of the total activity score in the example of ACE inhibitory activity will be described. FIG. 12 is a diagram illustrating an example of equations for calculating the total activity score.

Equation (1) is an example of an equation for a toxicity evaluation model for mixtures in the field of environmental toxicology. Pi denotes the proportion (concentration) of component i. ECx denotes the effective concentration at x %. Equation (1) is the sum (total value) of values obtained by dividing the concentration of each component by the toxicity value.

Equation (2) for the total activity score is a modification of Equation (1) assuming that toxicity and ACE inhibitory activity occur through similar mechanisms and using the concept of concentration addition in Equation (1). IC₅₀ denotes the half maximal inhibitory concentration of each component in its ACE inhibitory activity. Equation (2) is the sum (total value) of values obtained by dividing the concentration of each component by each half maximal inhibitory concentration.

When the total activity score in the example of ACE inhibitory activity is compared with the actually measured ACE inhibitory concentration of food as well, the coefficient of determination R², which indicates the correlation, is approximately 0.5, acknowledging a strong correlation, and it can be said that the evaluation by the total activity score is highly valid.

Multiple types of total activity scores, for example, a score for the preventive effect on a disease X and a score for the preventive effect on a disease Y may be calculated. For example, prevention scores for a disease X and a disease Y can be calculated by combining DPP-4 inhibition, antioxidant activity, SGLT1 activity inhibition, GLUT2 activity inhibition, FOXO1 activity inhibition, and the like.

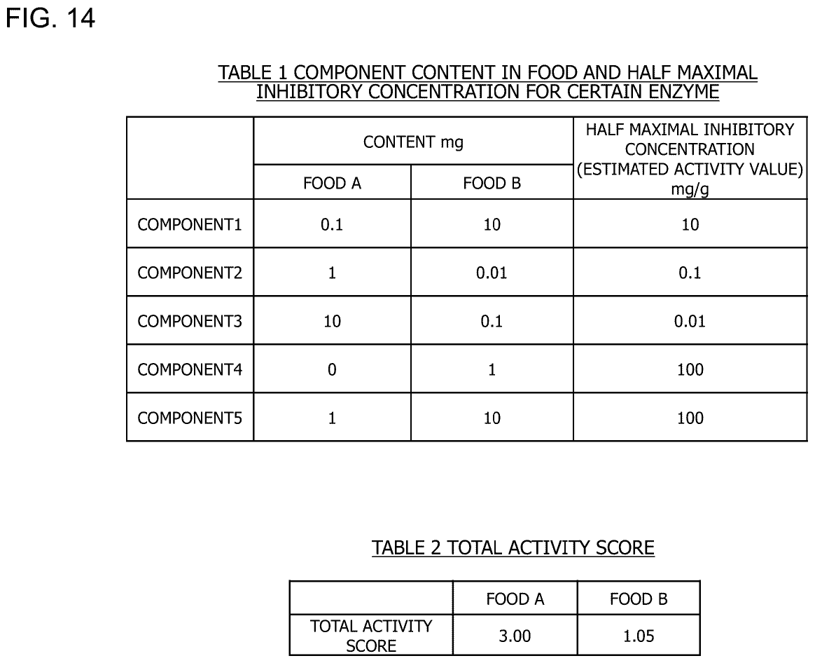

An example of the total activity score will be described. FIG. 14 is a diagram illustrating an example of the total activity scores of a food A and a food B. Table 1 in FIG. 14 is a table presenting the component contents in foods and the half maximal inhibitory concentrations for a certain enzyme, and presents the contents (milligrams) of components 1 to 5 contained in the food A and food B and the half maximal inhibitory concentrations of the respective components. Table 2 in FIG. 14 is a table presenting the total activity scores of the food A and food B calculated by the food evaluation device 100.

For example, the food evaluation device 100 substitutes the values in Table 1 into Equation (2) in FIG. 12 to obtain the following results.

Total activity score of food A=Log 10(0.1/10+1/0.1+10/0.01+0/100+1/100)=3.004

Total activity score of food B=Log 10(10/10+0.01/0.1+0.1/0.01+1/100+10/100)=1.049

For example, the food evaluation device 100 rounds off to the third decimal place to calculate the total activity score of the food A as 3.00 and the total activity score of the food B as 1.05. As a result, the total activity score of the food A is higher than the total activity score of the food B, and it can be evaluated that the food A is a food having a stronger degree of inhibitory activity for a certain enzyme.

### 2. Model Training Processing

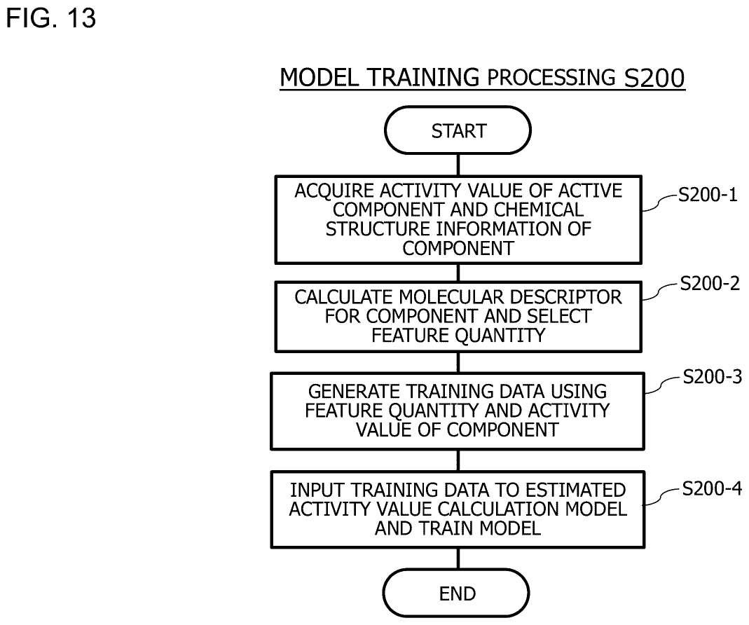

FIG. 13 is a diagram illustrating an example of a processing flowchart of model training processing S200. The food evaluation device 100 acquires chemical structure information and an activity value of an active component (hereinafter referred to as component in some cases) (S200-1). Here, the chemical structure information and activity value of the component to be acquired are, for example, literature data.

The food evaluation device 100 calculates a molecular descriptor for the component from the chemical structure information of the component and selects a feature quantity (S200-2). Here, the food evaluation device 100 selects, for example, a molecular descriptor that is presumed to be closely associated with the activity value of the component as a feature quantity. For example, the food evaluation device 100 may select a molecular descriptor that exhibits a remarkable feature as a feature quantity. The feature quantity is a value selected from molecular descriptors. In other words, the feature quantity is a type of molecular descriptor for which about 2000 types are calculated.

The food evaluation device 100 generates training data using the calculated feature quantities and activity values of components (S200-2). Then, the food evaluation device 100 inputs the training data to the estimated activity value calculation unit 22, causes the estimated activity value calculation unit 22 to execute training (S200-3), and ends the processing.

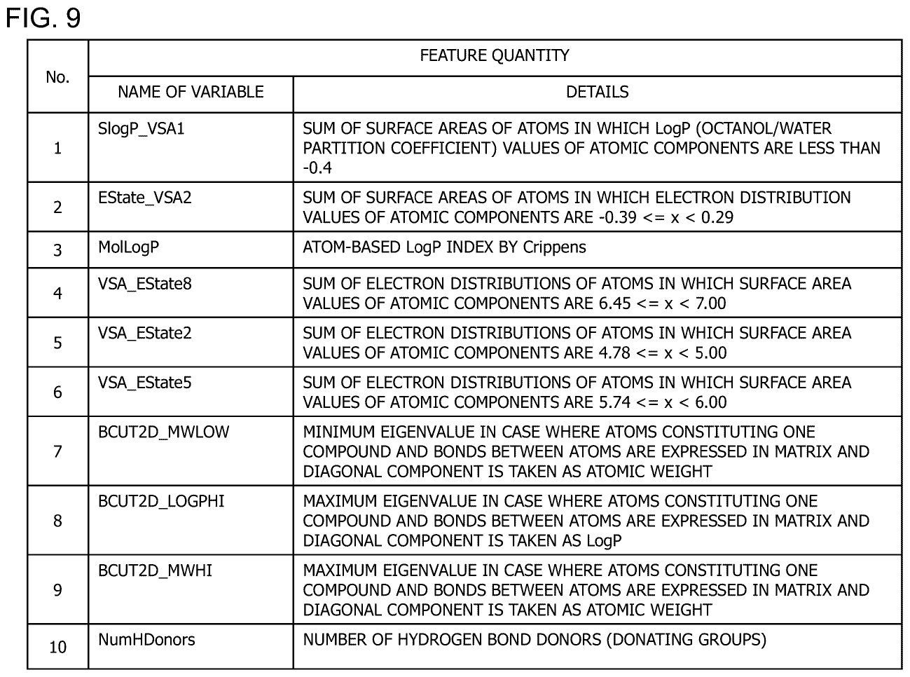

The feature quantities calculated in the example of ACE inhibitory activity will be described. FIG. 9 is a diagram illustrating an example of feature quantities to be calculated. There are, for example, 10 types of feature quantities calculated in the example of ACE inhibitory activity. The 10 types of feature quantities are feature quantities suitable for capturing features (tendencies) of the chemical structure of a peptide, which is the target component. In the example of ACE inhibitory activity, as illustrated in FIG. 9, molecular descriptors suitable for capturing features of the chemical structure of a peptide, which is the target component, are selected as feature quantities.

The numerical values illustrated in FIG. 9 are merely examples, and feature quantities using different numerical values may also be included. The food evaluation device 100 may calculate feature quantities other than the feature quantities illustrated in FIG. 9. It is preferable that the feature quantities calculated here are, for example, those according to the feature quantities used in model training. In addition to the same feature quantities, those according to the feature quantities used in model training include ones obtained by changing the numerical values in the calculation of the feature quantities. Those according to the feature quantities used in model training include feature quantities exhibiting similar tendencies in the chemical structure. In model training processing, a huge amount of data is obtained by generating training data from literature data. However, by limiting the feature quantities to be calculated, the effort and time required to generate training data can be reduced.

In addition to literature data, the teaching data may use activity values estimated using a learning model. For example, the food evaluation device 100 estimates the activity value of an unknown component (not found in literature data) using a learning model. The food evaluation device 100 may use this estimated activity value and the feature quantity of the unknown component as training data. This makes it possible to increase the types of training data and to train more deeply the learning model.

### OTHER EMBODIMENTS

The total activity score may be a value for animals other than humans. The total activity score may be used, for example, to evaluate feed for pets and livestock.

The total activity score may be a value per unit amount (for example, per gram) or a value according to the amount of the target food.

The food evaluation device 100 may calculate a plurality of total activity scores for one food. A plurality of total activity scores are calculated for each type of a plurality of diseases and injuries, for example, a total activity score for A (A is a disease name) prevention, a total activity score for B (B is a disease name) prevention, and a total activity score for C (C is a disease name) prevention. By calculating a plurality of total activity scores in this way, it becomes clear which disease or injury a certain food has a great effect on, for example, “the preventive effect on A and B is large but the preventive effect on C is small”. The user can select a food by referring to the plurality of total activity scores. For example, there is a case where a certain user is interested in the preventive effects on A and B but not in the preventive effect on C. In this case, the certain user may select a food mainly by referring to the total activity scores related to the preventive effects on A and B.

At the time of designing and production of food products, when a certain user prefers that the evaluation by the total activity score be a certain numerical value or more, a composition can be designed and produced, which is manufactured by adjusting components and in which the total activity score is calculated from estimated activity values and component amounts of the plurality of contained components, which are obtained by: analyzing a plurality of contained components that are contained or estimated to be contained in the composition and the component amounts of the plurality of contained components; calculating molecular descriptors related to the chemical structures of the contained components; and estimating the activity values of the contained components, based on the molecular descriptors, so that the evaluation by the total activity score is a certain numerical value or more.

When a certain user prefers that the evaluation by the total activity score be less than (or equal to or less than) a certain numerical value, a composition can be designed and produced, which is manufactured by adjusting components and in which the total activity score is calculated from estimated activity values and component amounts of the plurality of contained components, which are obtained by: analyzing a plurality of contained components that are contained or estimated to be contained in the composition and the component amounts of the plurality of contained components are analyzed; calculating molecular descriptors related to the chemical structures of the contained components; and estimating the activity values of the contained components, based on the molecular descriptors, so that the evaluation by the total activity score is less than (or equal to or less than) a certain numerical value.

In order to make the evaluation by the total activity score equivalent to a certain numerical value, a composition can be designed and produced, which is manufactured by adjusting the components and in which the total activity score is calculated from estimated activity values and component amounts of the plurality of contained components, which are obtained by analyzing a plurality of contained components that are contained or estimated to be contained in the composition and the component amounts of the plurality of contained components; calculating molecular descriptors related to the chemical structures of the contained components; and estimating the activity values of the contained components, based on the molecular descriptors, so that the evaluation by the total activity score is equivalent to a certain numerical value.

1. A composition evaluation device comprising:
an analyzer that analyzes a plurality of contained components that are contained or estimated to be contained in a composition and also analyzes component amounts of the plurality of contained components;
a molecular descriptor calculator that calculates molecular descriptors related to chemical structures of the contained components;
an activity value estimator that estimates activity values of the contained components, based on the molecular descriptors; and
a score calculator that calculates a total activity score of the plurality of contained components from the estimated activity values of the contained components and the component amounts, wherein
the activity value estimator estimates activity values of the contained components by a learned model that has learned using a feature quantity selected from the molecular descriptors for known components and a corresponding activity value as training data.

2. The composition evaluation device according to claim 1, wherein
the learned model detects a component having a chemical structure, which is similar to a chemical structure of the contained component, from learned components and calculates an activity value of the detected component as an activity value of the contained component.

3. The composition evaluation device according to claim 1, wherein
the total activity score is a logarithm of a sum of values obtained by dividing concentrations of respective contained components by activity values.

4. The composition evaluation device according to claim 1, wherein
the total activity score is a value obtained by summing numerical values obtained by multiplying activity values of the plurality of contained components by component amounts of the plurality of contained components.

5. The composition evaluation device according to claim 1, wherein
the learned model has learned using the feature quantity of a chemical structure of a component and the activity value, and
the feature quantity includes a feature quantity related to a sum of surface areas of atoms.

6. The composition evaluation device according to claim 1, wherein
the learned model has learned using the feature quantity of a chemical structure of a component and the activity value, and
the feature quantity includes a feature quantity related to a sum of electron distributions of atoms.

7. The composition evaluation device according to claim 1, wherein
the learned model has learned using the feature quantity of a chemical structure of a component and the activity value, and
the feature quantity includes a feature quantity related to an atom-based Log P index.

8. The composition evaluation device according to claim 1, wherein
the learned model has learned using the feature quantity of a chemical structure of a component and the activity value, and
the feature quantity includes a feature quantity related to eigenvalues in a case where atoms constituting one compound and bonds between atoms are expressed in a matrix and diagonal components are taken as atomic weights.

9. The composition evaluation device according to claim 1, wherein
the learned model has learned using the feature quantity of a chemical structure of a component and the activity value, and
the feature quantity includes a feature quantity related to a number of hydrogen bond donors.

10. The composition evaluation device according to claim 1, wherein
the known component includes the contained component estimated using the learned model, and
the corresponding activity value includes an activity value of the contained component estimated using the learned model.

11. The composition evaluation device according to claim 1, wherein
the contained component is a peptide,
the activity value is a half maximal inhibitory concentration for ACE (angiotensin-I converting enzyme),
the known component is a peptide of which an activity value is known, and
the score calculator calculates the total activity score, based on the calculated half maximal inhibitory concentration for ACE.

12. A composition evaluation method comprising:
an analysis step of analyzing a plurality of contained components that are contained or estimated to be contained in a composition and also analyzes component amounts of the plurality of contained components;
a descriptor calculation step of calculating molecular descriptors related to chemical structures of the contained components;
an estimation step of estimating activity values of the contained components, based on the molecular descriptors; and
a score calculation step of calculating a total activity score of the plurality of contained components from the estimated activity values of the contained components and the component amounts, wherein
in the estimation step, activity values of the contained components are estimated by using a learned model that has learned using a feature quantity selected from the molecular descriptors for known components and a corresponding activity value as training data.

13. A composition manufactured based on evaluation by a total activity score, wherein
the total activity score is calculated from estimated activity values of a plurality of contained components and component amounts, which are obtained by
analyzing the plurality of contained components that are contained or estimated to be contained in a composition and the component amounts of the plurality of contained components,
calculating molecular descriptors related to chemical structures of the contained components, and
estimating activity values of the contained components, based on the molecular descriptors.

14. A composition manufacturing method for manufacturing a composition, based on evaluation by a total activity score, wherein
the total activity score is calculated from estimated activity values of a plurality of contained components and component amounts, which are obtained by
analyzing the plurality of contained components that are contained or estimated to be contained in a composition and the component amounts of the plurality of contained components,
calculating molecular descriptors related to chemical structures of the contained components, and
estimating activity values of the contained components, based on the molecular descriptors.
